(19) United States
(12) Patent Application Publication
(10) Pub. No.: US 2017/0348444 A1
(43) Pub. Date: Dec. 7, 2017

(54) STERILIZER FOR PORTABLE ELECTRONIC DEVICE

(71) Applicant: Daekyu JANG, Hwaseong-si (KR)

(72) Inventor: Daekyu JANG, Hwaseong-si (KR)

(21) Appl. No.: 15/540,007

(22) PCT Filed: Dec. 23, 2015
(86) PCT No.: PCT/KR2015/014140

§ 371 Date: Jun. 26, 2017

(30) Foreign Application Priority Data
Dec. 24, 2014 (KR) 10-2014-0187894

(51) Int. Cl. A61L 2/08; A61L 2/26; H02J 7/00

(52) U.S. Cl. CPC A61L 2/084; H02J 7/0045; A61L 2/085; A61L 2/26; A61L 2202/11

(57) ABSTRACT

The present invention provides a sterilizer for a portable electronic device, comprising: a main body unit; a charge pin terminal unit coupled to one side surface of the main body unit and inserted into a charge slot of the portable electronic device; a cable coupling slot provided at the other side surface of the main body unit such that a charge terminal of a charge cable is inserted into the cable coupling slot; and a sterilizing LED module provided at one side surface of the main body unit, wherein LED rays emitted from the sterilizing LED module include visible rays, which are harmless to the human body.

### TECHNICAL FIELD

The present invention relates to a sterilizer for a portable electronic device and, more particularly, to a sterilizer for a portable electronic device, which is capable of being conveniently used due to a gender type, charging a portable electronic device, and simultaneously efficiently sterilizing for a portable electronic device by emitting a visible rays, which are harmless to the human body.

### BACKGROUND ART

In general, in most of portable electronic devices such as smartphone, a user performs input by directly touching a screen using his or her finger. Accordingly, many pathogenicity bacteria, such as various types of viruses or colon bacilli, may reproduce on a surface of a touch pad that is directly touched by a user's finger.

It is highly probable that many pathogenicity bacteria reproducing on the touch pad of a portable electronic device as described above may generate diseases harmful to the human body, such as causing a cold, food poisoning, tuberculosis and asthma, because they are introduced into the mouse and nose through a finger that has touched the touch pad.

In order to solve such a problem, there were proposed technologies for inserting a smartphone, etc. into a separate sterilizing apparatus or sterilizing a smartphone by coupling a holder and a sterilizer, such as No. 10-1230474 and No. 20-0470126.

However, such conventional technologies have low portability because equipment for sterilizing a smartphone, etc. is relatively large and ultraviolet rays generated upon the sterilization being performed may be harmful to the human body. Accordingly, there is a need for a new alternative.

### SUMMARY OF THE INVENTION

The present disclosure provides a sterilizer for a portable electronic device in which is capable of being conveniently used due to a gender type, charging a portable electronic device, and simultaneously efficiently sterilizing a portable electronic device by emitting a visible rays, which are harmless to the human body.

An exemplary embodiment of the present disclosure provides a sterilizer for a portable electronic device that may include a main body unit; a charge pin terminal unit coupled to one side surface of the main body unit inserted into a charge slot of a portable electronic device; a cable coupling slot provided at the other side surface of the main body unit such that a charge terminal of a charge cable is inserted into the cable coupling slot; and a sterilizing LED module provided at one side surface of the main body unit, wherein LED rays emitted from the sterilizing LED module include visible rays, which are harmless to the human body.

An one side surface of the main body unit in which the sterilizing LED module is provided may be depressed and formed in a concave groove shape.

LED ray emitted from the sterilizing LED module may further include infrared rays.

A battery may be embedded in the main body unit, and the sterilizing LED module may be supplied with power from the battery embedded in the main body unit and may light emitting LED.

According to the present disclosure can be conveniently used due to a gender type, can charge a portable electronic device, and can also efficiently sterilize for a portable electronic device by emitting a visible rays, which are harmless to the human body.

### BRIEF DESCRIPTION OF THE DRAWINGS

FIG. 1 is a diagram showing a perspective view of a sterilizer for a portable electronic device according to an exemplary embodiment of the present invention.

FIG. 2 is a diagram showing a rear perspective view of a sterilizer for a portable electronic device shown in FIG. 1.

FIG. 3 is a diagram showing a side sectional view of a sterilizer for a portable electronic device shown in FIG. 1.

FIG. 4 is a diagram showing an example in which the sterilizer for a portable electronic device shown in FIG. 1 is coupled to a portable electronic device and used.

FIG. 5 is a diagram showing an example of another exemplary embodiment of the present invention.

### BEST MODE FOR EMBODIMENTS

The present invention provides a sterilizer for a portable electronic device, includes a main body unit;

a charge pin terminal unit coupled to one side surface of the main body unit and inserted into the charge slot of the portable electronic device;

a cable coupling slot provided at the other side surface of the main body unit such that a charge terminal of a charge cable is inserted into the cable coupling slot; and

a sterilizing LED module provided at the periphery of the charge pin terminal unit,

wherein LED rays emitted from the sterilizing LED module include visible rays, which are harmless to the human body.

### DETAILED DESCRIPTION OF THE EMBODIMENTS

The present invention will be described more fully herein after with reference to the drawings, in which exemplary embodiments of the invention are shown.

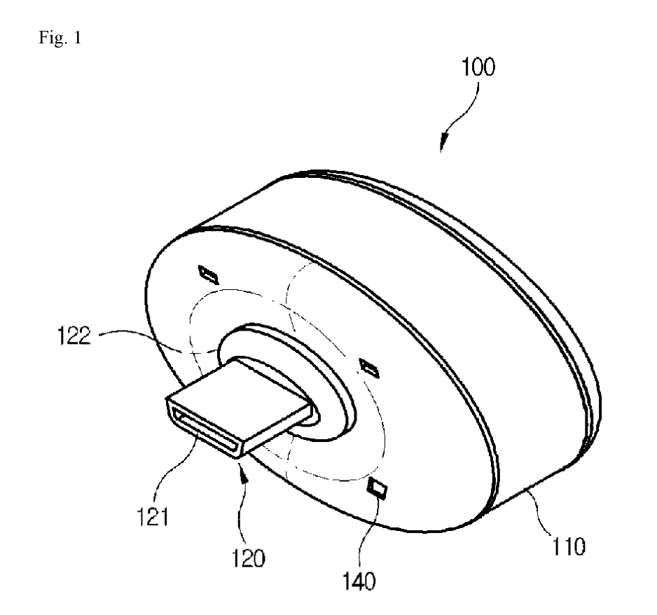

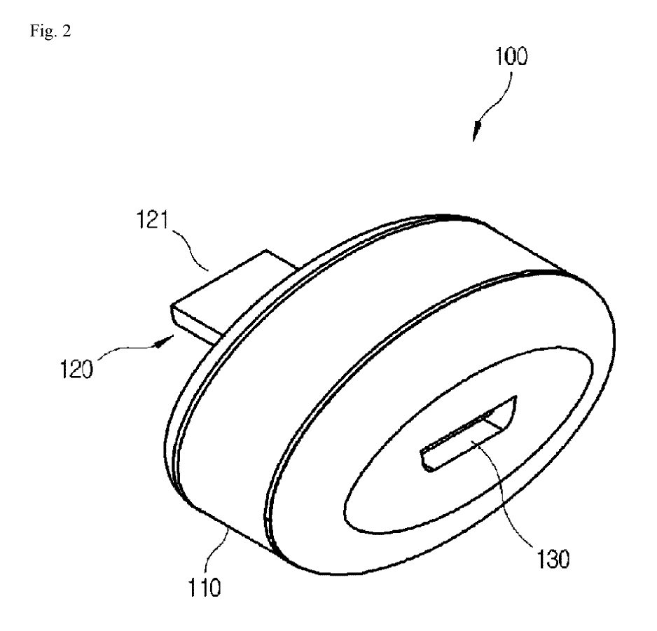

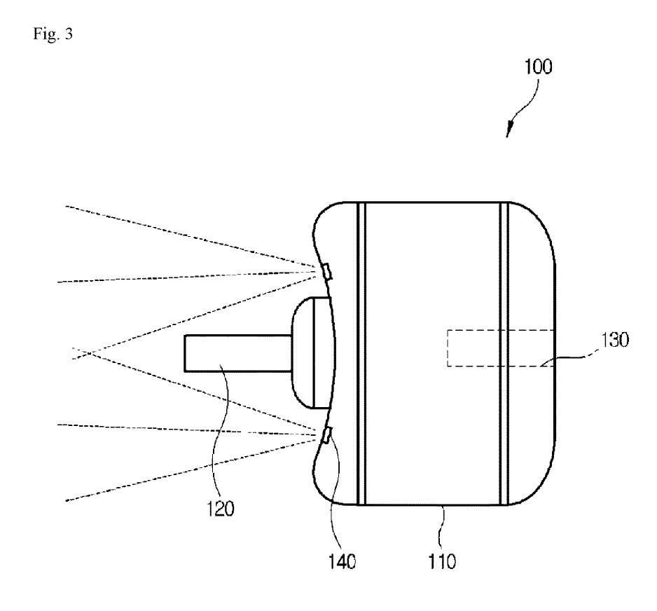

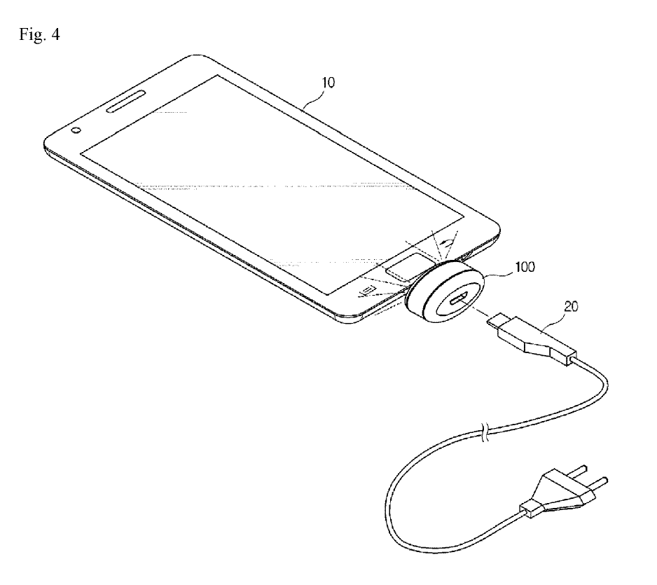

FIG. 1 is a diagram showing a perspective view of a sterilizer for a portable electronic device according to an exemplary embodiment of the present invention, FIG. 2 is a diagram showing a rear perspective view of the sterilizer for a portable electronic device shown in FIG. 1, FIG. 3 is a diagram showing a side sectional view of the sterilizer for a portable electronic device shown in FIG. 1, and FIG. 4 is a diagram showing an example in which the sterilizer for a portable electronic device shown in FIG. 1 is coupled to a portable electronic device and used.

As shown, the sterilizer for a portable electronic device 100 according to an embodiment of the present invention includes a main body unit 110 of a cylindrical shape having both end clogged, a charge pin terminal unit 120 coupled to one side surface of the main body unit 110, a cable coupling slot 130 for cable coupling provided at the other side of the main body unit 110, and a sterilizing LED module 140 provided at the periphery of the charge pin terminal unit 120.

The main body unit 110 provides an installation space for the elements, and preferably has one side surface depressed and formed in a concave groove shape and the other side surface formed to have a flat surface. A driving circuit (not shown) for the sterilizing LED module may be provided within the main body unit. The size of the mainbody unit 110 is not limited to the illustrated size, but may be fabricated in a greater size or smaller size depending on the necessity of sterilizing power.

The charge pin terminal part 120 includes a charge pin 121 and a terminal body 122 and is coupled to the main body unit 110.

The cable coupling slot 130 is formed on the other side surface of the mainbody unit 110 and may be provided to be horizontal to the charge pin terminal unit 120.

The charge terminal of a charge cable 20 is inserted into the cable coupling slot 130, so a current for charging is output toward the charge pin terminal unit 120.

A plurality of pins is provided in the cable coupling slot 130 for cable coupling in accordance with the number of charge pins 121 of the charge pin terminal unit 120. For example, if the number of charge pins is 5 as in an android phone, it is preferred that the number of pins provided in the cable coupling slot 130 for cable coupling is 5. Accordingly, in the case of the iphone, it is preferred that each of the number of pins of 8 provided in the cable coupling slot is 8 may be formed. However, the present invention is not essentially limited to such structures. Accordingly, the charge pin 121 and the cable coupling slot 130 for cable coupling may be formed in a form having different numbers of pins, such as a pin conversion gender, and may be used depending on required needs.

A plurality of the sterilizing LED module 140 is provided at the periphery of the charge pin terminal unit 120 and one side of surface the main body unit 140, and radiates LED rays to a portable electronic device 10 coupled to the charge pin terminal unit 120, such as a smartphone, etc. The LED ray may be preferably a visible ray having a wavelength of 380˜760 nm. Such a wavelength band is a visible ray band and harmless to the human body unlike ultraviolet rays, thereby being capable of guaranteeing safety in use. However, such a visible ray may have a low sterilizing effect for a short period of time compared to ultraviolet rays, but the present invention has been proposed with the intention of additionally performing sterilization on the portable electronic device 10 simultaneously with long-time charging. If charging of 3˜4 hours or more is performed, a corresponding sterilizing effect can be sufficiently accomplished.

In addition, the sterilizing LED module 140 further includes an LED that emits infrared rays of 760 nm˜1 mm. If an LED that emits infrared rays is included as described above, there is an advantage in that sterilizing efficiency by a visible rays LED can be improved because heat is generated.

The sterilizing LED module 140 have been illustrated as being 4 in the illustrated an embodiment, but the number of sterilizing LED modules 140 may be adjusted in various ways, if necessary.

The LED of the sterilizing LED module 140 are formed to have their radiation angles of 110˜120 degrees. Although the LED are mounted on one side of the portable electronic device 10, the front and back sides of the portable electronic device can be sufficiently radiated. However, it is preferred that one side surface of the main body unit 140 is formed in a round shape as shown in FIG. 3 so that the LED rays emitted from the sterilizing LED module 140 are further concentrated toward the portable electronic device, such as a smartphone, etc.

That is, as in the illustrated an embodiment, if four sterilizing LED module 140 are provided, two of the four sterilizing LED module 140 are provided in an upper column and the remaining two sterilizing LED modules 140 are disposed in a lower column, and coupling with the portable electronic device is performed, the sterilizing LED module 140 in the upper column may radiate LED rays to the front of the portable electronic device and the sterilization LED module 140 in the lower column may radiate LED rays to the back of the portable electronic device. In this case, if the sterilizing LED module 140 are not provided in a round shape, but are vertically provided, some of the LED rays may be lost without being directed toward a smartphone, etc. In order to prevent such a loss, the LED module 140 may be fixed in a round shape, thereby being capable of maximizing sterilization efficiency by minimizing the leakage of light.

An action of the sterilizer for a portable electronic device configured as described above is described. First, as in FIG. 4, when the charge terminal of the charge cable 20 is coupled to the cable coupling slot 130 formed on one side of the sterilizer 100 according to the present invention so that a current for charging is transferred to the charge pin terminal unit 120, a charging operation is performed as usual and a sterilizing LED module driving circuit (not shown) provided within the body part 110 operates to make the plurality of sterilizing LED module 140 emit light using some of the current for charging. Accordingly, the plurality of sterilizing LED module 140 emits a visible ray harmless to the human body or a visible ray and infrared rays harmless to the human body, so the sterilizing and charging of the portable electronic device 10, such as a smartphone etc, can be performed at the same time by the plurality of sterilizing LED module 140.

Furthermore, the sterilizer 100 is a small-sized gender type and can be conveniently carried or kept, thereby being capable of significantly increasing use convenience.

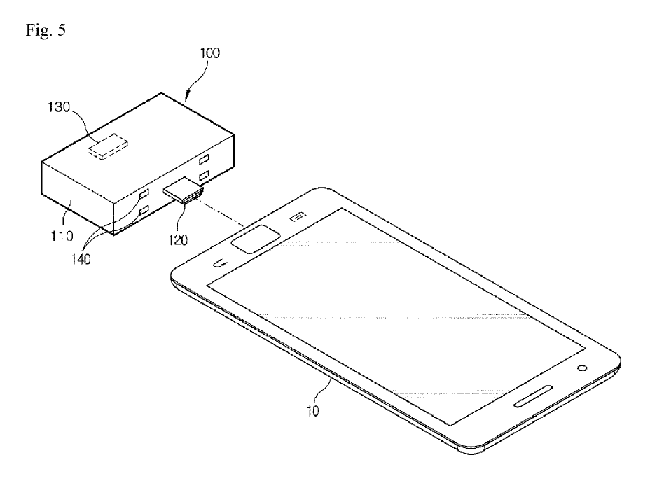

FIG. 5 is a diagram showing an example of another exemplary embodiment of the present invention. A sterilizer for a portable electronic device 100 according to the illustrated embodiment has a portable battery pack form and additionally includes a portable battery function. A charge pin terminal unit 120 is formed on one side of a main body unit 110, and a cable coupling slot 130 for cable coupling is formed on the other side of the main body unit 110. The formation locations are not essentially limited to the illustrated locations. The sterilizing LED module 140 are provided at the periphery of the charge pin terminal unit 120, and most of the elements are the same as those of the aforementioned embodiments.

In the present exemplary embodiment, however, a portable battery (not shown) is embedded in the main body unit 110. The sterilizing LED module 140 is configured to be supplied with power from the portable battery (not shown) embedded in the main body unit 110 and to made light emitting LED.

Accordingly, when the charge pin terminal unit 120 is coupled to a portable electronic device 10, the portable electronic device 10 is charged and at the same time, the sterilization of the portable electronic device 10 is made possible. According to the exemplary embodiment of the present, there is an advantage in that the portable electronic device can be sterilized in the state in which a separate charge cable has not been connected.

The present invention may be modified and implemented in various manners by applying a plurality of sterilizing LED module to the periphery of the charge pin terminal unit of a charge device for a portable electronic device of a different form, such as a stand dock for charging, in addition to the illustrated embodiments.

As described above, the present invention is not limited to the described embodiments and it is evident to a person having ordinary skill in the art that the present invention may be modified and transformed in various ways without departing from the spirit and range of the present invention. Accordingly, it will be said that such modified examples and transformed examples belong to the claims of the present invention.

### INDUSTRIAL APPLICABILITY

The present invention may be used in an industry regarding a sterilizer for a portable electronic device.

What is claimed is:

1. A sterilizer for a portable electronic device, comprising:
a main body unit;
a charge pin terminal unit coupled to one side surface of the main body unit and inserted into a charging slot of a portable electronic device;
a cable coupling slot provided at the other side surface of the main body unit such that a charge terminal of a charge cable is inserted into the cable coupling slot; and
a sterilizing LED module provided at a periphery of the charge pin terminal unit,
wherein LED rays emitted from the sterilizing LED module comprises a visible rays, which are harmless to the human body.

2. The sterilizer for a portable electronic device of claim 1, wherein the one side surface of the main body unit in which the sterilizing LED module is provided may be depressed and formed in a concave groove shape.

3. The sterilizer for a portable electronic device of claim 1, wherein the LED ray emitted from the sterilizing LED module further comprises infrared rays.

4. The sterilizer for a portable electronic device of claim 1, wherein:
a battery is embedded in the main body unit, and
the sterilizing LED module is supplied with power from the battery embedded in the main body unit and made light emitting LED.
